(19) United States
(12) Patent Application Publication
(10) Pub. No.: US 2011/0306669 A1
(43) Pub. Date: Dec. 15, 2011

(54) RAPID ONSET AND SHORT TERM MODAFINIL COMPOSITIONS AND METHODS OF USE THEREOF

(75) Inventors: Eyal S. Ron, Lexington, MA (US); Neal M. Farber, Waban, MA (US); Daniel E. Katzman, Newton, MA (US); Elkan R. Gamzu, Newton, MA (US)

(73) Assignee: NeuroHealing Pharmaceuticals, Inc.

(21) Appl. No.: 13/214,893

(22) Filed: Aug. 22, 2011

Related U.S. Application Data
(63) Continuation of application No. 11/988,695, filed on Jan. 11, 2008, now abandoned.
(60) Provisional application No. 60/701,281, filed on Jul. 21, 2005.

(51) Int. Cl. A61K 31/165; A61P 25/00

(52) U.S. Cl. 514/618

(57) ABSTRACT

Compositions are described that comprise a modafÊnil component that is a combination of the d- and l-enantiomers of modafinil and wherein the modafÊnil component is greater than 50% by weight d-modafÊnil for use in promoting or enhancing the state of wakefulness, alertness, and/or central nervous system stimulation in an individual.

### CROSS-REFERENCE TO RELATED APPLICATIONS

This application claims priority to U.S. Provisional Application No. 60/701,281, filed Jul. 21, 2005.

### BACKGROUND OF THE INVENTION

A variety of drugs are known for their ability to stimulate or enhance various activities of the mammalian central nervous system (CNS) and/or to promote or enhance an individual's state of wakefulness or alertness. Examples of such drugs having one or more such pharmacological activities include such well known and diverse drugs as methylxanthines (e.g., caffeine, theophylline, theobromine), nicotine, amphetamines, methylphenidates (e.g., RITALIN®, Novartis), and modafinil.

Modafinil (2-[(diphenylmethyl)sulfinyl]acetamide) is a relatively recent addition to the list of drugs known to promote CNS stimulation, wakefulness, and/or alertness. Modafinil is structurally distinct from various groups of classic CNS stimulants and also has a distinctly different mode of action that has yet to be fully elucidated. The compound was originally identified as a member of a genus of acetamide derivatives developed by the Laboratoire Louis Lafon in the 1970s (see, e.g., U.S. Pat. No. 4,177,290; U.S. Pat. No. 5,612,379).

Modafinil is a racemic compound with a chiral center at its sulfur atom. Modafinil molecules exist as either of two optically active forms, i.e., “d-modafinil” (dextrorotatory enantiomer) and “l-modafinil” (levorotatory enantiomer). Accordingly, preparations of modafinil are available as the optically inactive racemic mixture (racemate, racemic modification), i.e., having equal amounts of the two enantiomers. Currently, modafinil is approved for use as a wakefulness-promoting agent for use in the treatment of excessive daytime sleepiness (EDS) in individuals who suffer from narcolepsy (see, e.g., Wong et al., J. Clin. Pharmacol., 39: 30-40 (1999); U.S. Reissue Pat. No. RE37,516 E). The commercially available formulation of modafinil is the orally administrable tablet PROVIGIL® (Cephalon, Inc., West Chester, Pa.) containing 100 mg or 200 mg of modafinil as the racemic mixture. An advantage of using modafinil is that modafinil is generally considered to have fewer side effects or side effects that are more readily treated than those associated with other drugs, such as the stimulant amphetamine and structurally related compounds that are known to exert an effect on the CNS.

The optical enantiomers of modafinil have similar pharmacological actions in animals. Both d-modafinil and l-modafinil have been shown to have the same pharmacological activity as the modafinil racemic compound in mice, however, pharmacokinetic studies of the racemic compound have shown that the l-modafinil has a half-life (T_{1/2}) in the human body of approximately 10-14 hours compared with 3-4 hours for the d-modafinil. In addition, the elimination of d-modafinil has been reported to be three times faster than the l-modafinil. As a result of the difference in half-life and rate of clearance, the use of racemic modafinil results in significant differences in circulating levels of the two enantiomers. The amount of d-modafinil in the circulation can be three times less and of a shorter duration than that of l-modafinil. After a single oral dose, racemic modafinil is readily absorbed, reaching maximum plasma concentrations at 2-4 hours after administration. See, e.g., Wong et al., J. Clin. Pharmacol., 39: 30-40 (1999); Wong et al., J. Clin. Pharmacol., 39: 281-288 (1999); Robertson et al., Clin Pharmacokinet., 42: 123-137 (2003); and Dinges et al., Curr. Medical Research and Opinions, 22: 159-169 (2006).

The pure d-modafinil enantiomer has not been previously studied in humans.

Accordingly, for treating the narcoleptic patient, the currently available, commercial preparations of modafinil provide a relatively slow onset time (long Tₘₐₓ), e.g., within 2 to 3 hours, and a relatively prolonged period of enhanced wakefulness per unit dose of more than about 11 hours. However, it is clear that the desired effect exerted on the CNS by drugs in currently available pharmaceutical preparations, including those of modafinil, typically will persist far beyond the period of time during which an individual may require the benefit of enhanced wakefulness or CNS stimulation. In many cases, an undesirable persistence of action on the CNS may be manifested in the individual complaining of retaining excessive alertness or agitated state such that the individual may not be able to remain calm during otherwise normal daily activities or of being unable to enter a normal restful sleep cycle leading to sleep deprivation. The only way to avoid such unsatisfactory interference with normal daily activities and a normal sleep cycle is for the individual to restrict the dose of the drug as well as the time at which the dose is administered, however, such restrictions may also limit the time during which the individual would desire the benefit of the wakefulness-promoting activity of the drug.

There are a variety of conditions and situations wherein an individual could benefit from a much shorter period of wakefulness-, alertness-, and/or CNS stimulation-promoting activity than provided by currently available drug formulations. Accordingly, needs remain for means and methods that provide an individual with a relatively rapid onset and a relatively short term of such pharmacological activities so that the individual may not also experience interference with the ability to subsequently carry out various tasks or enter into a normal sleep cycle in the absence of such activities.

### BRIEF SUMMARY OF THE INVENTION

The invention described herein solves the above problems by providing compositions that provide an individual with a relatively rapid onset (short Tₘₐₓ) and relatively short duration (short T_{1/2}) of an enhanced state of wakefulness, alertness, and/or of central nervous system (CNS) stimulation.

In one embodiment, there is provided a composition for promoting or enhancing the state of wakefulness, alertness, and/or stimulation of the CNS in an individual (human or other mammal) comprising a modafinil component that is a combination or mixture of the d- and l-enantiomers of modafinil, wherein greater than 50% by weight of the modafinil component is the d-enantiomer of modafinil (d-modafinil). Such compositions may comprise a modafinil component that is greater than 50% (by weight) and less than 100% (by weight) d-modafinil. In order of increasing preference, compositions described herein may comprise a modafinil component that is greater than 50% (by weight) and up to 60% (by weight) d-modafinil, greater than 60% and up to 70% d-modafinil, greater than 70% and up to 80% d-modafinil, greater than 80% and up to 90% d-modafinil, greater than 90% and up to 95% d-modafinil, and greater than 95% and up to 99% d-modafinil.

In another embodiment, a composition useful in the invention may comprise a modafinil component that is greater than 50% and up to 100% d-modafinil.

In another embodiment, a composition described herein may comprise a modafinil component that is greater than 0% by weight to less than 50% by weight the l-enantiomer of modafinil (l-modafinil).

In yet another embodiment, a composition useful in the invention may comprise a modafinil component that is essentially 0% l-modafinil, i.e., essentially 100% d-modafinil.

Preferably, the state of enhanced wakefulness, alertness, and/or CNS stimulation provided to an individual by a single dose of a composition described herein lasts for less than about 11 hours, more preferably less than 10 hours. Depending on the particular ratio of d- and l-enantiomers employed as the modafinil component of compositions described herein, an individual may obtain a period of wakefulness-, alertness-, or of CNS stimulation-promoting activity ranging from about 1 to less than 10 hours. Particularly preferred are compositions of the invention that contain more than 90% (by weight) d-modafinil or, for some uses, substantially only d-modafinil, to provide a period of enhanced CNS stimulation, wakefulness, and/or alertness of from about 1 to about 4 hours.

In a preferred embodiment, compositions described herein are formulated in a delivery form that provides a rapid onset of one or more of the pharmacological activities of modafinil in an individual, including, but not limited to, orally dissolvable films, fast dissolving tablets, a solution, and mucoadhesive microparticles. A particularly preferred route of administration of compositions described herein is sub-lingual.

In another embodiment, compositions described herein further comprise one or more other agents that provide a beneficial feature to the composition including, but not limited to, a pharmaceutically acceptable carrier, a taste-masking agent, a flavoring agent, a drug different from modafinil that affects the central nervous system, an antimicrobial agent, a plasticizing agent, a buffering agent, a lubricating agent, a preservative, an inert filler agent, a hydrogel, a coloring agent, an enhancer of absorption or transport across mucous membranes, and combinations thereof.

Compositions described herein may be administered to an individual either parenterally or non-parenterally. Preferably, compositions are administered by a route other than via ingestion into the stomach and intestinal tract. Such preferred routes of administration of a composition described herein include sublingual, buccal, nasal, pulmonary, and rectal. In a particularly preferred embodiment, a composition described herein is administered sublingually.

Compositions described herein may be used in any of a variety of situations where an individual may benefit from a relatively short period of enhanced wakefulness or alertness or CNS stimulation in order to counteract fatigue and enhance concentration e.g., during the performance of various tasks, while operating machinery, while operating a vehicle, during a period of learning new subject matter, and during a period of participating in a neurorehabilitation program, without disrupting or interfering with the ability of the individual to subsequently resume other activities or to rest or enter into normal sleep in the absence of the previously enhanced state of wakefulness, alertness, and/or CNS stimulation.

A preferred method of treating an impaired neurological function in an individual comprises:

administering to the individual a composition described herein comprising a modafinil component, wherein the modafinil component comprises from greater than 50% to 100% by weight d-modafinil, and

engaging the individual in a neurorehabilitation program comprising one or more neurostimuli.

In another embodiment of a method of treating an impaired neurological function of an individual, the individual is taken through multiple (two or more) rounds of administration of a composition described herein followed by participation in a neurorehabilitation program.

### DETAILED DESCRIPTION OF THE INVENTION

The invention provides compositions and methods for counteracting fatigue, for promoting or enhancing the state of wakefulness, alertness, or central nervous system (CNS) stimulation in an individual. Compositions described herein comprise a modafinil component, wherein the modafinil component comprises greater than 50% (by weight) of the d-enantiomer of modafinil (d-modafinil) and wherein the compositions are so formulated as to provide an individual with both a relatively rapid onset of and relatively short duration of the wakefulness-, alertness-, and/or CNS stimulation-promoting activity of the modafinil component. Accordingly, compositions described herein provide an individual with greater control over the timing and duration of a beneficial modafinil effect (e.g., enhanced CNS stimulation, wakefulness, and/or alertness) along with the benefit of minimal interference with the individual's ability to subsequently engage in other activities or to enter into a normal sleep cycle in the absence of the previously experienced modafinil effect.

In order that the invention may be more clearly understood, the following terms are defined.

A “drug” refers to any compound or composition that has a pharmacological activity. Thus, a “therapeutic drug” is a compound or composition that can be administered to an individual to provide a desired pharmacological activity to the individual. A “prophylactic drug” is a compound or composition that can be administered to an individual to prevent or provide protection from the development in an individual of an undesired or harmful condition or disorder. A drug may have prophylactic as well as therapeutic uses.

Terms such as “parenteral”, “parenterally”, and the like, refer to routes or modes of administration of a compound or composition to an individual other than along the alimentary canal. Examples of parenteral routes of administration include, without limitation, subcutaneous (s.c.), intravenous (i.v.), intramuscular (i.m.), intra-arterial (i.a.), intraperitoneal (i.p.), transdermal (absorption through the skin or dermal layers), nasal (“intranasal”; absorption across nasal mucosa), or pulmonary (e.g., inhalation for absorption across the lung tissue), vaginal, direct injections or infusions into body cavities or organs other than those of the alimentary canal, as well as by implantation of any of a variety of devices into the body (e.g., of a composition, depot, or device that permits active or passive release of a compound or composition into the body).

The terms “non-parenteral”, “non-parenterally”, “enteral”, “enterally”, “oral”, “orally”, and the like, refer to administration of a compound or composition to an individual by a route or mode along the alimentary canal. Examples of enteral routes of administration include, without limitation, oral, as in swallowing solid (e.g., tablet) or liquid (e.g., syrup) dosage forms, sublingual (i.e., administration under the tongue for absorption through the mucosal membranes lining the floor of the mouth), buccal (absorption through the mucosal membranes lining the cheeks), nasojejunal or gastrostomy tubes (delivery into the stomach), intraduodenal administration, as well as rectal administration (e.g., suppositories for release of a drug composition into and absorption by the lower intestinal tract of the alimentary canal). Sublingual and buccal routes of administration are considered particularly well suited for producing a rapid onset of drug action while avoiding passage of the drug through the gut for absorption.

The term “brain injury” is a general term used to refer to a condition that results in central nervous system (CNS) damage, irrespective of the physiopathological source. The most frequent origins of brain injury include stroke, traumatic brain injury (TBI), encephalitis, multiple sclerosis, major organ failure, and degenerative diseases (e.g., Parkinson's Disease). Traumatic brain injury (TBI) and stroke are among the most frequently occurring and widely known events that can cause brain injury and an associated impairment of one or more neurological functions. Among the variety of cases of TBI diagnosed each year in the United States and around the world are vehicle accidents, such as involving a car, motorcycle, or bicycle. Stroke represents the leading cause of disability in adulthood. Patients that suffer a stroke can present disabilities associated with impairment of any of a variety of neurological functions as described above, including, but not limited to, motor function (e.g., impairments in strength, dexterity, swallowing), sensory functions (e.g., anesthesia, propioceptive deficits), speech function (e.g., aphasia, dysarthria), and cognitive functions (e.g., deficiency in planning, short and long term memory loss (amnesia), working memory loss, attention deficits, spatial attention deficits).

“Neurological function” refers to a function of the body of an individual that requires normal functioning neural transmission. Neurological functions of an individual that may be impaired and, therefore treated according to the invention, include, without limitation, functions that are primarily sensory (e.g., light sensing, tactile sensing, hot-cold sensing), primarily cognitive (e.g., reading, memory, comprehension, reasoning, learning), locomotor (or simply, “motor”) functions that are primarily based on movement (e.g., directed body movements, walking, maintaining balance), or a combination thereof (e.g., coordination of cognitive and motor functions as required in communicating, use of tools, operating machines, self-care, and other activities). Impaired neurological functions may also be referred to by the name for the corresponding neurological deficit or disorder, e.g., aphasia, dysarthria, amnesia, paralysis, anesthesia, propioceptive deficits, and the like. Any of a variety of disorders or conditions may lead to the impairment of one or more neurological functions of an individual including but not limited to brain injury (see, above), brain cancer, brain surgery, epilepsy, Parkinson's Disease, multiple sclerosis, pain, sleep disorders, neuro-endocrine disorders, neuromuscular disorders, childhood developmental disorders, and psychiatric disorders.

“Neurorehabilitation”, as used herein, refers to any rehabilitation program that may be used for the purpose of treating or improving one or more neurological functions that may have been impaired (e.g., lost or diminished) in an individual as the result of an injury to the brain or other portion of the nervous system. Neurorehabilitation regimens useful in the invention provide one or more neurostimuli (e.g., exercises, tasks, light stimulation, audio stimulation, visual stimulation, tactile stimulation) designed to restore or enhance one or more impaired neurological functions of an individual and may include classical physical therapy exercises. Such neurostimuli are routinely repeated by the individual, and the effect on the impaired function can be monitored and assessed by one trained in neurorehabilitation. Thus, such exercises or tasks may include forms of physical therapy to promote development of an impaired motor function; exercises or tasks for improving aspects of cognitive functions, e.g., memory, reading, recognition of objects, comprehension, response to commands, and the like; and exercises or tasks designed to improve a combination of motor and cognitive functions, e.g., speech, writing, operating machines, and the like. Neurorehabilitation regimens may also include electrical/magnetic stimulation regimens (e.g., trans-cranial magnetic stimulation (TMS), deep brain stimulation (DBS), electroconvulsive therapy; see, also, U.S. Pat. No. 6,463,328). The goal of neurorehabilitation is to improve one or more neurological functions that were impaired due to injury in an individual and, thereby, advance the individual toward increased participation and independence in self-care, mobility, and/or employment.

The term “modafinil” is synonymous with benzhydrylsulfinyl acetamide and 2-[(diphenylmethyl)sulfinyl]acetamide as described in U.S. Pat. No. 5,612,379; U.S. Pat. No. 6,489,363; and U.S. Reissue Pat. No. RE37,516 (the teachings of which are incorporated herein by reference). It is also understood that the terms “modafinil”, “benzhydrylsulfinyl acetamide”, and “2-[(diphenylmethyl)sulfinyl]acetamide” encompass the various organic and inorganic acid salt forms of the above structure.

As noted above, modafinil molecules exist as either of two different enantiomers (d- and l-enantiomers) that do not interconvert. Thus, modafinil may be produced as an optically inactive racemic mixture (also referred to as a “racemate” or “racemic modification”). Individual enantiomers may be synthesized by various published protocols (see, e.g., U.S. Pat. No. 4,927,855, providing individual synthetic protocols for making l- and d-enantiomers; Prisinzano et al., Tetrahedron Asymmetry, 15: 1053-1058 (2004), providing a synthetic protocol specifically for making d-modafinil). Individual enantiomers of modafinil may also be resolved from the racemate (see, e.g., Donovan et al., Ther. Drug Monit., 25(2): 197-202 (2003)).

Modafinil is approved for the treatment of excessive daytime sleepiness (EDS) in individuals who suffer from narcolepsy (see, e.g., Wong et al., J. Clin. Pharmacol., 39: 30-40 (1999); U.S. Reissue Pat. No. RE37,516 E). The commercially available formulation of modafinil is the orally administrable tablet PROVIGIL® (Cephalon, Inc., West Chester, Pa.) that contains 100 mg or 200 mg of modafinil as the racemic mixture (racemate, racemic modification). The d- and l-enantiomers of modafinil have the same pharmacological activity, but different pharmacokinetics. The modafinil racemate has a half-life (T_{1/2}) of about 15 hours, similar to the circulating half-life of l-modafinil (T_{1/2} of approximately 13-16 hours), whereas d-modafinil is eliminated from the human body at an approximately three-fold faster rate than l-modafinil (T_{1/2} of approximately 3 hours) (see, e.g., Wong et al., J. Clin. Pharmacol., 39: 30-40 (1999); Wong et al., J. Clin. Pharmacol., 39: 281-288 (1999)). Accordingly, the currently available pharmaceutical compositions of modafinil are so formulated as to provide an individual with a relatively prolonged period of enhanced wakefulness so that the individual is more alert and more able to better perform cognitive and locomotor tasks throughout the daytime.

The pharmacological activities of modafinil clearly include promoting CNS stimulation as well as promoting wakefulness and alertness in humans and other mammals, however, the precise pharmacological mechanism(s) by which modafinil effects such activities has not been conclusively elucidated. For example, modafinil has been reported to stimulate the CNS as an adrenergic agonist resulting in increased locomotor activity and/or enhanced wakefulness (see, e.g., Duteil et al., Eur. J. Pharmacol., 180: 49-58 (1990), Saletu et al., Int. J. Clin. Pharm. Res., 9: 183-195 (1989), Jouvet et al., Encephale, 17:187-195 (1991), or that it may work by modulating GABAergic tone (Ferraro et al., Eur J Pharmacol., 306: 33-39 (1996)). Wisor et al. (J. Neurosci, 21:1787-1794 (2001)) have shown that modafinil increases extracellular dopamine and that dopamine transporter gene knock-out mice were unresponsive to the action of modafinil; again, indicative of the ability of modafinil to exert a stimulatory effect on the CNS.

In contrast, the information regarding modafinil in the package insert of PROVIGIL® (Cephalon) as approved by the United States Food and Drug Administration states that modafinil appears to be neither a direct nor indirect α₁-adrenergic agonist nor to exert any sympathomimetic activity.

The exact mechanism of action of racemic modafinil is unclear, although in vitro studies have shown it to inhibit the reuptake of dopamine by binding to the dopamine reuptake pump, and lead to an increase in extracellular dopamine. Modafinil activates glutamatergic circuits while inhibiting GABA. Modafinil is thought to have less potential for abuse than other CNS stimulants due to the absence of any significant euphoric or pleasurable effects. It is possible that modafinil acts by a synergistic combination of mechanisms including direct inhibition of dopamine reuptake, indirect inhibition of noradrenalin reuptake in the VLPO and orexin activation. Modafinil has partial alpha 1B-adrenergic agonist effects by directly stimulating the receptors.

A particularly problematic side effect of currently available modafinil compositions, which contain exclusively l-modafinil or the modafinil racemate, is that the period of enhanced wakefulness, alertness, or CNS stimulation is so prolonged as to interfere with an individual's ability to subsequently engage in other activities, including the benefit of a normal sleep cycle. Accordingly, currently available compositions of modafinil are clearly not suited for use according to the invention wherein an individual desires to benefit from a relatively short period of an enhanced state of wakefulness, alertness, or CNS stimulation and subsequently carry on other activities or enter a normal sleep cycle in the absence of any substantial pharmacological activity of modafinil.

A composition or method described herein as “comprising” one or more named elements or steps is open-ended meaning that the named elements or steps are essential, but other elements or steps may be added within the scope of the composition or method. To avoid prolixity, it is also understood that any composition or method described as “comprising” (or “comprises”) one or more named elements or steps also describes the corresponding, more limited, composition or method “consisting essentially of” (or “consists essentially of” the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and close-ended composition or method “consisting of” (or “consists of”) the named elements or steps to the exclusion of any other unnamed element or step. In any composition or method disclosed herein, known or disclosed equivalents of any named essential element or step may be substituted for that element or step.

The meaning of other terms will be evident by the context of use and, unless otherwise defined, have the meaning commonly understood by persons skilled in neurology, pharmacology, and neurorehabilitation.

### Compositions and Delivery Forms

As noted above, compositions of the invention comprise a modafinil component that is a combination or mixture of the d- and l-enantiomers of modafinil, wherein the modafinil component is greater than 50% by weight the d-enantiomer of modafinil (d-modafinil). Preferably, the modafinil component of a composition described herein is a combination or mixture of d- and l-modafinil and is greater than 50% (by weight) and less than 100% (by weight) of d-modafinil. In order of increasing preference, compositions described herein may comprise a modafinil component that is greater than 50% (by weight) and up to 60% (by weight) d-modafinil, greater than 60% and up to 70% d-modafinil, greater than 70% and up to 80% d-modafinil, greater than 80% and up to 90% d-modafinil, greater than 90% and up to 95% d-modafinil, and greater than 95% and up to 99% d-modafinil. For some uses, a composition described herein may comprise a modafinil component that is greater than 50% and up to 100% d-modafinil.

Compositions described herein may also comprise a modafinil component that is a combination or mixture of d-modafinil and l-modafinil and is greater than 0% (by weight) to less than 50% (by weight) l-modafinil. In other embodiments, a composition useful in the invention may comprise a modafinil component that is essentially 0% l-modafinil, i.e., essentially 100% d-modafinil.

Mixtures of enantiomers that may be used as modafinil components of compositions described herein include those that exhibit a dextrorotatory specific optical activity relative to the optically inactive modafinil racemate.

Another feature of preferred compositions described herein is that the modafinil component is formulated as to provide an individual with a relatively rapid onset of a state of enhanced wakefulness, alertness, or CNS stimulation for a period of time that is shorter than the period provided by previously available compositions containing only the l-enantiomer or the racemate of modafinil. Accordingly, the compositions described herein provide an individual with finer control over the duration of the effect of modafinil on the central nervous system (CNS) such that, with appropriate scheduling of doses, an individual may both obtain the benefit of a period of enhanced wakefulness, alertness, and/or CNS stimulation and subsequently enter into and enjoy the benefit of normal sleep.

As with any drug, it is understood that a composition of the invention must deliver at least a threshold amount of modafinil that is effective to exert an effect on the CNS to promote or enhance the state of wakefulness, alertness, and/or CNS stimulation in an individual. The determination of such a minimal effective dose in a particular composition is readily made using methods known in the art for formulating CNS stimulants and wakefulness and alertness promoting pharmaceutical compositions. For example, enhanced wakefulness or alertness may be detected and assessed in an individual using standard methods (e.g., observations, inquiries, parameters) of wakefulness and alertness as currently employed by persons skilled in the art of formulating and manufacturing commercially available preparations of modafinil or other wakefulness promoting drugs. Computer programs are available that provide accurate assessments of an individual's fatigue and alertness to perform cognitive and/or locomotor (physical) tasks (e.g., Automated Neuropsychological Assessment Metrics (“ANAM”) as developed by the Naval Computer and Telecommunications Station, Pensacola, Fla.). Generally, compositions of the invention may be formulated to contain a dose of total modafinil (i.e., sum of all enantiomers) in the range of from about 10 milligrams (mg) to about 600 mg of modafinil and, more preferably, about 50 mg to about 200 mg of modafinil.

As the total amount of modafinil increases in a composition, the intensity of CNS stimulation, wakefulness, and/or alertness promoting activity is, in general, expected to increase, but the length of time for which such activity persists is determined mainly by the circulating half-lives of the modafinil d- and l-enantiomers and the relative amounts of each enantiomer in a particular composition of the invention. Owing to the fact that compositions described herein always contain a modafinil component in which d-modafinil is the major (i.e., greater than 50% by weight) or only (100% by weight) enantiomeric species, the compositions are so formulated as to provide a period of enhanced CNS stimulation, wakefulness, and/or alertness to an individual that persists for a period of time that is shorter than current commercially available compositions that are so formulated to contain only the l-enantiomer or the modafinil racemic mixture and that provide a relatively prolonged period (e.g., greater than 11 hours) of relief from excessive daytime sleepiness (EDS) in narcoleptic individuals. Preferably, compositions according to the invention are formulated so as to provide an individual with a period of enhanced CNS stimulation, wakefulness, and/or alertness for less than about 11 hours, more preferably less than 10 hours. Depending on the particular ratio of d- and l-enantiomers employed in the modafinil component of compositions described herein, an individual may obtain a period of CNS stimulation, wakefulness, and/or alertness promoting activity ranging from about 1 to less than 10 hours. Particularly preferred are compositions of the invention that comprise a modafinil component comprising more than 90% (by weight) d-modafinil or substantially only d-modafinil and that provide a modafinil effect for a period of about 1 to about 4 hours.

Compositions comprising modafinil as described herein may be formulated in any of a variety of solid, semi-solid, or liquid delivery (“dosage”) forms. Generally, compositions of the invention may be formulated for administration to an individual according to standard pharmaceutical protocols and texts (e.g., Remington's Pharmaceutical Sciences, 18th ed., Alfonso R. Gennaro, ed. (Mack Publishing Co., Easton, Pa. 1990)). Compositions of the invention preferably comprise a pharmaceutically acceptable carrier as well as any of a variety of other compounds that may be used in preparing a pharmaceutical composition for administration by a particular mode or route, i.e., parenteral or oral. By “pharmaceutically acceptable” is meant a material that is not biologically, chemically, or in any other way, incompatible with body chemistry and metabolism and also does not adversely affect the desired, effective activity of the modafinil component or any other component in a composition described herein.

Modafinil is essentially water insoluble (water solubility of about 0.4 mg/ml). Accordingly, preparation of compositions according to the invention may employ various dry methods of preparation (see below) or the use of pharmaceutically acceptable organic solvents. Nevertheless, in the course of preparing various compositions, it may be useful or necessary to use one or more pharmaceutically acceptable aqueous carriers including, but not limited to, water, physiological saline, and aqueous buffers.

The pharmaceutical compositions of this invention for oral administration may include, but are not limited to, liquids, lozenges, tablets, pills, capsules, caplets, oleaginous suspensions, syrups, elixirs, and sublingually administrable films. Capsules, tablets, pills, and caplets may also be formulated for rapid disintegration (“fast dissolving”). In the case of tablets for oral use, carriers, which are commonly used include lactose and corn starch. Lubricating agents, such as magnesium stearate, may also be added.

Compositions of the invention are preferably prepared in a delivery form to provide an onset of CNS stimulation, wakefulness, and/or alertness promoting activity that is more rapid than currently available tablet forms of modafinil that are swallowed (ingested) and absorbed via the gastrointestinal tract. Particularly preferred for use in the invention are compositions that deliver an effective amount of a modafinil component across mucosal membranes (mucosa) to underlying blood vessels without the need for ingestion and subsequent passage into the stomach and intestines. Such tissues include the mucosal membranes lining the bottom of the mouth (e.g., sublingual tissue), the cheeks of the mouth (e.g., buccal administration), the nasal passages, the vagina, and the rectum. Relatively rapid delivery and onset of activity are possible because such tissues provide minimal barriers to the underlying blood vessels so that the drug can enter those blood vessels for delivery to the brain. For example, preferred compositions according to the invention may be so formulated for administration to the sublingual tissue, where they rapidly dissolve to release an effective amount of the modafinil component that is then rapidly absorbed by the mucosal tissue into the underlying blood vessels and, thereby, enter the systemic circulation directly. Sublingual administration also has the advantage that the drug bypasses the gastrointestinal tract and the liver, thereby avoiding inactivation by hepatic metabolism. As much as 90% of modafinil delivered by ingestion of PROVIGIL® tablets is known to be eliminated by the liver in humans (see, PROVIGIL® package insert, Cephalon).

Compositions of the invention may be formulated in any of a variety of sublingually administrable delivery forms, including fast dissolving tablets films (“filmstrips”), solutions, and suspensions. Particularly preferred are sublingually administrable film (or “filmstrip”) compositions that provide a relatively rapid delivery of the modafinil component to an individual. Various types of films for delivering a drug have been described (see, e.g., U.S. Pat. No. 6,177,096; U.S. Pat. No. 5,700,478; U.S. Pat. No. 6,756,051; U.S. Pat. No. 6,552,024). Such films are thin solid compositions that dissolve or disintegrate when they come in contact with the saliva. Films may become bioadhesive upon wetting, which permits them to readily adhere under the tongue, to the tongue, gums, or cheek. This bioadhesive property of films serves as an effective means of preventing the film from being swallowed and, thereby, restricts release of the modafinil component from the film to the mucosal tissues of the mouth, such as the sublingual tissue, for rapid absorption through the relatively thin mucosal tissue lining the mouth and into underlying blood vessels (as opposed to via the gastrointestinal tract). Thus, sublingually administered compositions comprising a modafinil component as described herein provide an especially rapid delivery of the modafinil to the CNS to provide a rapid onset of enhanced CNS stimulation, wakefulness, and/or alertness.

Preferably, film compositions useful in the invention have a disintegration rate in the human mouth in the range of 1 second to 1200 seconds, more preferably 1 second to 600 seconds, even more preferably 1 second to 300 seconds, still more preferably 1 second to 150 seconds, and most preferably 1 second to 60 seconds. Particularly preferred are bioadhesive “fast-dissolving” film compositions that dissolve in less than about 1 minute, and preferably, in 1-10 seconds when administered sublingually or buccally. Preferred bioadhesive “slow-dissolving” films may take more than 1 minute, more preferably, 5 to 30 minutes, to dissolve when applied sublingually or buccally.

Film compositions comprising a modafinil component as described herein may also contain any of a variety of other pharmaceutically acceptable ingredients (“excipients”) that contribute to producing a film. Such excipients may include, but are not limited to, a buffering agent, a plasticizing agent, a stabilizing agent, a taste-masking agent, a flavoring agent, a breath freshening agent, a coloring agent, an antiseptic, an inert filler agent, a preservative, and combinations thereof. Preferably, films comprising a modafinil component as described herein will have a thickness in the range of less than 0.25 millimeters (mm) to 5 mm. Particularly preferred are films that are less than 0.25 mm in thickness.

Tablets that disintegrate or dissolve rapidly in the patient's mouth are convenient for providing young children, the elderly, and patients with swallowing difficulties, the benefit of the modafinil compositions described herein. Such tablets are also convenient where potable liquids are not available. For such formulations, the small volume of saliva is usually sufficient to result in tablet disintegration in the oral cavity. The medication (modafinil) can then be absorbed partially or entirely into the systemic circulation from blood vessels in the sublingual mucosa, or it can be swallowed as a solution to be absorbed from the gastrointestinal tract. As noted above, the sublingual route usually produces a faster onset of action than orally ingested tablets, and the portion absorbed through the sublingual blood vessels bypasses the hepatic first-pass metabolic processes (see, e.g., Birudaraj et al. J. Pharm. Sci., 94:70-78 (2005); Ishikawa et al., Chem. Pharm. Bull. (Tokyo) 49: 230-232 (2001); and Price et al., Obstet. Gynecol., 89: 340-345 (1997)).

Various techniques may be used to formulate rapidly disintegrating (fast dissolving) tablets (Allen L V. Rapid-dissolve technology: an interview with Loyd V. Allen. Int. J. Pharm. Techno., 7: 449-450 (2003); Fu et al., Crit. Rev. Ther. Drug Carrier Syst., 21: 433-476 (2004)). Fast disintegrating tablets technologies are often based on lyophilization, molding, sublimation, and compaction. The fast disintegrating tablet properties can be enhanced by such approaches as spray-drying, moisture treatment, sintering, use of sugar-based disintegrants and taste-masking technologies (Fu et al, Crit. Rev. Ther. Drug Carrier Syst., 21(6): 433-76 (2004)). For example, direct compression, one of these techniques, requires the incorporation of a superdisintegrant into the formulation, or the use of highly water-soluble excipients to achieve fast tablet disintegration. Direct compression does not require the use of water or heat during the formulation procedure and is the ideal method for moisture- and heat-labile medications. However, the direct compression method is very sensitive to changes in the type and proportion of excipients and in the compression forces, when used to achieve tablets of suitable hardness without compromising the rapid disintegration characteristics. Unique packaging methods, such as strip-packaging, may be used to compensate for the problem of extreme friability of such rapidly disintegrating tablets. Ideal excipient proportions and other related parameters using a superdisintegrant in order to formulate durable fast-disintegrating tablets for oral administration have been explored (see, e.g., Watanabe et al., Biol. Pharm. Bull., 18: 1308-1310 (1995); Bi et al., Chem. Pharm. Bull. (Tokyo), 44: 2121-2127 (1996)).

Accordingly, a fast disintegrating tablet is a particularly useful format as it provides a means for enhanced release of modafinil from the formulation for rapid absorption by the sublingual mucosa blood vessels. Such tablets can be made by selecting the appropriate pharmaceutical excipients in the correct proportion, in combination with optimal manufacturing techniques and compression parameters.

Another preferred formulation that provides a more efficient and desirable delivery of the modafinil component than swallowing tablets is a nasally (“intranasally”) administrable delivery form that delivers the modafinil component to the intranasal mucosa or a form that delivers modafinil to the lungs for absorption to underlying blood vessels. Intranasally administrable forms include, but are not limited to, formulations that may be applied directly to or sprayed (nebulized) into the nasal passages and also microparticles that may be suspended in a carrier for applying to or spraying into the intranasal passages (see, e.g., Cilurzo et al., Eur. J. Pharm. Sci., 24(4): 355-361 (2005)). Typically, such modes of administration require that a composition be provided in the form of a solution, liquid suspension, or powder, which is mixed with a gas (e.g., air, oxygen, nitrogen, etc., or combinations thereof) so as to generate an aerosol or suspension of droplets or particles. Intranasally and pulmonary administrable compositions are prepared employing techniques known in the art and may include saline, a preservative (e.g., benzyl alcohol), and/or other solubilizing or dispersing agents known in the art. Intranasally administrable formulations may also comprise one or more agents that enhance transport and absorption of the modafinil component across the nasal mucosa.

A composition comprising modafinil according to the invention may also comprise any of a number of various pharmaceutically acceptable carriers, or excipients known in the art that may provide one or more beneficial pharmacological properties, including but not limited to, more efficient delivery of the modafinil component to the central nervous system, less objectionable or less painful administration to an individual, and/or longer storage of compositions (i.e., enhanced shelf-life). Accordingly, pharmaceutical compositions of this invention may include, without limitation, sweeteners, ion exchangers, alumina, aluminum stearate, lecithin, serum proteins (e.g., human serum albumin, etc.), buffering agents (e.g., phosphates, citrate, glycine, sorbic acid, potassium sorbate, and the like), partial glyceride mixtures of saturated vegetable fatty acids, water, salts or electrolytes (e.g., protamine sulfate, disodium hydrogen phosphate, potassium hydrogen phosphate, sodium chloride, zinc salts, and the like), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, lanolin, a taste-masking agent, a flavoring agent, and combinations thereof.

Flavoring agents and flavor enhancers make the dosage form more palatable to the patient, particularly in the cases where absorption takes place in the oral mucosa during the residence time in the oral cavity.

Common flavoring agents and flavor enhancers for pharmaceutical products that may be included in the composition of the present invention include, but are not limited to, maltol, vanillin, ethyl vanillin, menthol, citric acid, fumaric acid ethyl maltol, tartaric acid, and combinations thereof.

To mask the taste of modafinil, sweeteners and/or flavoring agents having the capability of masking the flavor of the modafinil compound may be used. Such taste-masking agents useful in the compositions described herein include, but are not limited to, one or more sweeteners selected from the group consisting of calcium saccharinate, ammonium cyclamate, ammonium glycirhizinate, aspartame, glucose and glucitols such as inositol, mannitol, sorbitol, or dulcitol, and/or at least one flavoring agent selected from the group consisting of natural or artificial fruit flavors. Taste-masking agents may be present in compositions described herein in a variety of ranges, such as in an amount ranging from about 1.0 mg to about 10.0 mg (such as 4.0 mg to 8.0 mg of aspartame), from about 100.0 mg to about 400.0 mg (such as 200.0 mg to 350.0 mg of glucose), from about 200 mg to about 800 mg (such as 300 mg to 700 mg of sorbitol), and from about 5.0 mg to about 50.0 mg (such as 10.0 mg to 30.0 mg of any of a variety of natural or artificial fruit flavors) per unit dosage.

The consistency and viscosity of a composition of the invention may be controlled by incorporating one or more polymers or hydrogels that absorb water and thereby produce gels of varying viscosity. Hydrogels suitable for use in pharmaceutical preparations are well known in the art (see, e.g., Handbook of Pharmaceutical Excipients, (The American Pharmaceutical Association and The Pharmaceutical Society of Great Britain (1986)); Handbook of Water-Soluble Gums and Resins, (ed. R. L. Davidson) (McGraw-Hill Book Co., New York 1980)). Hydrogels that may be useful in various compositions described herein include, but are not limited to, hydroxypropyl cellulose, hydroxypropyl methyl cellulose, sodium carboxymethylcellulose (“CMC”), polyacrylic acid, poly(methyl methacrylic acid) (“PMMA”), and combinations thereof. When present in the compositions of this invention, the hydrogel(s) preferably comprises from about 0.1% to about 50% by weight to volume (w/v) of the composition.

Compositions of this invention may also be administered in the form of suppositories for releasing the modafinil component into a body cavity other than the mouth or stomach, e.g., for rectal or vaginal administration. Such compositions can be prepared by mixing various desired pharmacologically active components, such as modafinil and other pharmacologically active agents, with a suitable non-irritating excipient that is solid at room temperature but liquid at body temperature and, therefore, after insertion into a space (e.g., vaginal or rectal space) will melt and release the modafinil component that can be absorbed across the mucosal tissue and into underlying blood vessels. Such excipients may include, but are not limited to, cocoa butter, beeswax, and polyethylene glycols.

Pharmaceutical compositions of the invention may be packaged in a variety of ways that are appropriate to the dosage form and mode of administration. These include but are not limited to vials, bottles, cans, packets, ampoules, cartons, flexible containers, inhalers, and nebulizers. Such compositions may be packaged for single or multiple administrations from the same container. For individuals with motor neuron disorders, especially useful are packages that are easy to open. For individuals who must work at nighttime, packages may be used that are easy to identify and open in low light conditions.

Kits may comprise a modafinil composition as described herein prepared in a form for delivery by an appropriate route along with instructions for administering the composition. For example, a kit may comprise a modafinil-containing composition in dry powder or lyophilized form, optionally along with an appropriate diluent (e.g., buffer, saline, etc.), which are to be combined shortly before administration by a particular route according to the accompanying instructions.

Various antimicrobial agents may also be used in compositions of the invention to prevent degradation and contamination. Such commonly used antimicrobial agents include phenol, benzyl alcohol, meta-cresol, methyl paraben, propyl paraben, benzalconium chloride, and benzethonium chloride. Such agents are present at concentrations that will prevent the growth of bacteria, fungi, and the like, but are non-toxic when administered to the intended individual.

As may be required by applicable regulatory standards, compositions described herein are prepared consistent with good manufacturing practices that are currently used in the pharmaceutical industry and that are well known to the skilled practitioner, Further, as may be required, sterile compositions are prepared in accordance with industry and regulatory standards using any of a variety of methods for sterilizing pharmaceutical compositions including, without limitation, ultrafiltration, autoclaving, dry and wet heating, exposure to gases such as ethylene oxide, exposure to liquids, such as oxidizing agents, including sodium hypochlorite (bleach), exposure to high energy electromagnetic radiation, such as ultraviolet light, x-rays or gamma rays, and exposure to ionizing radiation. Choice of method of sterilization, where required, will be made by the skilled practitioner with the goal of effecting the most efficient sterilization that does not significantly alter the desired pharmacological activity of the modafinil component or any other component of a composition intended for administration to an individual. Ultrafiltration procedures may be particularly useful in the sterilization process for pharmaceutical compositions that are aqueous solutions or suspensions.

### Uses

Compositions comprising modafinil as described herein find use in any of a variety of situations in which an individual may desire or would benefit from a relatively rapid onset and short period of enhanced wakefulness or alertness or CNS stimulation without disrupting or interfering with the ability of the individual to subsequently resume other activities, to rest, or to enter into normal sleep in the absence of the previously enhanced state of wakefulness, alertness, or CNS stimulation. Such situations include, without limitation, whenever it is desirable for an individual to have a means to counteract fatigue and enhance concentration, e.g., during the performance of specific tasks or activities, during the operation of machinery, during the operation of a vehicle, during a period of learning new subject matter, and during a period of participation in a neurorehabilitation program to treat or improve one or more neurological functions that may have been impaired in the individual.

Compositions described herein are particularly useful to maintain, promote, or enhance CNS stimulation, wakefulness, and/or alertness in an individual in situations where fatigue or a diminished ability to concentrate (decreased attentiveness) by an individual presents a risk of serious harm to life or property. For example, sustained human performance is critical to successful completion of tasks or activities carried out in many healthcare institutions (e.g., emergency care, intensive care, surgery) and governmental agencies (e.g., national defense, aerospace, air traffic control), as well as during the course of operating machinery or a vehicle, particularly a motor vehicle (e.g., an automobile, a truck, a motorcycle, an aircraft, a boat, a ship, a train, a streetcar, an armored vehicle, etc.).

Fatigue and loss of attentiveness are especially of concern for operators of motor vehicles who are homeward bound after completing work during a nightshift. Such workers have been shown to be exceptionally susceptible to accidents due to loss of attentiveness or consciousness while commuting home in the morning after working through the night. The Federal Motor Carrier Safety Administration (United States Department of Transportation) has initiated programs to reduce truck fatalities that specifically employ practices and technology to monitor and counteract fatigue of truck drivers. Composition as described herein that provide a relatively rapid onset of and a relatively short duration of CNS stimulation, wakefulness, and/or alertness enhancing activity of modafinil are particularly useful in such situations as these compositions provide an individual with a finer degree of control over the period of modafinil's effect than previously possible such that, with appropriate scheduling of doses, the individual may subsequently enter into and enjoy the benefit of a normal sleep cycle without interference by an undesirably prolonged or lingering modafinil activity.

Compositions comprising a modafinil component as described herein may also be used in treating patients including, but not limited to, promoting a patient's recovery from anesthesia and in various treatment regimens for patients with attention deficit disorder (ADD) or attention deficit hyperactivity disorder (ADHD).

Compositions described herein also find use in neurorehabilitation programs and regimens to treat one or more neurological functions that may have been impaired (i.e., lost or diminished) in an individual. Neurorehabilitation programs typically provide one or more neurostimuli, which may include various tasks or exercises, designed to restore or strengthen one or more impaired neurological functions in an individual. The effectiveness of a neurorehabilitation program for improving an impaired neurological function of an individual may be monitored and assessed by trained personnel using any of a variety of standard scales including, but not limited to, the Disability Rating Scale (DRS) (Rappaport et al., Arch. Phys. Med. Rehabil., 63: 118-123 (1982)), the Functional Independence Measure™ (FIM™) assessment scale (Guide for the Uniform Data Set for Medical Rehabilitation (including the FIM™ instrument), Version 5.1 (State University of New York at Buffalo, Buffalo, N.Y., 1997)), and the Rivermead Motor Assessment Scale (Winward et al., Clin. Rehabil., 16(5): 523-533 (2002)).

Attempts have been made to improve the effectiveness of traditional neurorehabilitation programs for treating an impaired neurological function in an individual by administering to the individual a drug known to have a pharmacological activity that affects neural function or that stimulates the CNS, and then having the individual participate in the activities of the program during the period of the drug's activity. Such attempts have met with mixed results depending on the drug employed. For example, Scheidtman et al. (Lancet, 358(9284): 787-790 (2001)) have reported improved outcome for recovery of impaired motor function in stroke patients when patients where administered levodopa prior to participating in a physiotherapy program, although the authors also noted that long term exposure to levodopa has various undesirable side effects. In contrast, Treig et al. (Clin. Rehabil., 17(6): 590-599 (2003)) reported that administration of the CNS stimulant D-amphetamine to individuals did not significantly enhance results of physiotherapy to improve motor function.

Administration of modafinil to individuals who participate in a neurorehabilitation program can improve the effectiveness of the program to treat an impaired neurological function (see, e.g., PCT Publication No. WO2004/082624). As noted above, compositions described herein provide an individual with a more rapid onset and shorter term of modafinil activity than previously possible using commercial formulations containing the modafinil racemate or l-modafinil. Accordingly, a composition as described herein may also be used in methods for treating an impaired neurological function in an individual with the added benefit that the individual can subsequently engage in other activities or enter into sleep without interference by a prolonged or persistent modafinil effect. The ability to enter and enjoy a normal sleep cycle promotes long-term memory and neural plasticity (see, e.g., Walker et al., Neuron, 44: 121-133 (2004)), both of which are considered to be beneficial to enhancing the effectiveness of a neurorehabilitation program to improve or restore an impaired neurological function in an individual. Moreover, as the term of modafinil activity provided to an individual by compositions described herein may be relatively short (e.g., 1 to 4 hours), with proper scheduling and dosing, an individual may be able to participate in multiple (2 or more) rounds of a neurorehabilitation program in a single day where each round of participation is separated by a rest period in the absence of modafinil activity. Engaging an individual in multiple daily rounds of a neurorehabilitation regimen is also considered to be beneficial to enhancing the effectiveness of a neurorehabilitation program on an individual.

In order to more fully illustrate the invention, the following non-limiting examples are provided.

### EXAMPLES

### Example 1

### Preparation of d-modafinil Formulations

The synthesis of (d)-(+)-modafinil has been described in the literature (see, e.g., Prisinzano et al., Tetrahedron Asymmetry, 15: 1053-1058 (2004); U.S. Pat. No. 4,927,855 (“Lafon synthesis”)). In accordance with the Lafon synthesis, the intermediate carboxylic acid was converted to the diastereomic salt mixture with (+) alpha-methylbenzylamine. The diastereomers were separated and the appropriate chiral acid liberated from the salt form. The acid was converted to the methyl ester via esterification and reacted with ammonia/methanol solution to yield d-modafinil. The enantiomeric purity was in excess of 98%-99%.

Initial formulation tests of modafinil (racemate) and pure d-modafinil revealed a bitter taste. Therefore, the pharmaceutical formulation included one or more taste-masking ingredients. The d-modafinil was mixed with various taste-masking agents, including pulverized mints, breathe fresheners, and natural and artificial flavorings.

The synthesized d-modafinil was compounded into a composition containing sugar, spearmint flavor, cinnamon flavor, gum arabic, gelatin, corn syrup, and dyes that could be administered sublingually. Formulations containing 65 mg, 100 mg, and 200 mg of d-modafinil were prepared.

### Example 2

### Double Blind Study of Sub-Lingual d-Modafinil Composition in a Normal Human Volunteer

The goal of this test was to confirm the suitability of the sublingual formulation and to ascertain if the purported short acting d-modafinil test article, under conditions of being very tired near to bedtime, had an affect on wakefulness.

The test subject was given three vials: one vial containing the base formulation (as in Example 1) to test the taste and delivery means, and two coded vials. One of the coded vials contained 100 mg of d-modafinil formulation, and the second coded vial contained placebo (an equivalent amount of formulation).

The subject was instructed to place the contents of the test formulation under the tongue, to allow the formulation to dissolve over two minutes, and to rinse any residual material with some water.

### Results

Subject reported that the taste of the base formulation was pronounced, but tolerable.

At 11:15 p.m. in the evening, the contents of one coded vial was placed under the tongue, allowed to dissolve over two minutes, and any residual was rinsed with some water. A strong taste remained for some time. The test subject then reported reading in bed, dozing on and off for approximately 1.5 hours. After the room was darkened, the subject reported sleeping lighter, and awakening at least once at 3:15 a.m. during the night.

On a following evening at 11:15 p.m., the contents of the second coded vial was similarly placed under the tongue, allowed to dissolve over two minutes under the tongue and rinsed. The test subject reported reading in bed until 12:50 a.m., then darkening the room and sleeping undisturbed all night until the morning.

At both evenings the test subject reported being equally very tired. Approximately one week before these tests, the same subject took 200 mg racemic modafinil (Provigil®) at 10 p.m. and reported a very pronounced effect, essentially being keep awake through the night to 5 a.m.

Before unblinding the test articles, the subject recorded that one of the test articles was active, but neither test substance kept him awake as strongly or as long as the 200 mg racemic modafinil. After unblinding the test articles, it was confirmed that the coded vial with the reported activity contained 100 mg d-modafinil.

### Example 3

### Double Blind Crossover Study of Sub-Lingual d-Modafinil in Normal Human Volunteers

Two subjects received a set of coded vials containing either 200 mg of d-modafinil formulated according to Example 1 or an equivalent placebo formulation.

The subjects were given the following instructions:
Start testing at approximately the same time every evening, one hour before bedtime.
Randomly select one of the coded vials each day.
Open the vials and place the powder under your tongue.
Allow the powder to dissolve slowly for approximately 1-2 minutes.
After the powder is fully dissolved, you may drink some water.
Record observations in a Visual Analogue Scale (VAS) describing the difficulty to fall asleep.

Visual Analog Scale (0 to 3)

| Normal | | | Kept awake, |
|---|---|---|---|
| ability to sleep | | | difficult to fall asleep |
| 0 | 1 | 2 | 3 |

0: fell asleep as usual
1: slight, noticeable change
2: noticeable change
3: extreme change (kept awake most of night)

Results from Subject 1

Observations from vial with code no. 33 (later described as placebo): VAS=0. No effect noticed. Went to bed at usual time and fell asleep as usual.

Observations from the second coded vial (later described as containing 200 mg of d-modafinil): VAS=1. It took longer than usual to fall asleep. Subject awoke several times during the night and felt more awake.

Results from Subject 2

Observations from one coded vial (later described as placebo): VAS=0. No effect noticed. Subject went to bed at usual time and fell asleep as usual.

Observations from the second coded vial (later described as containing 200 mg of d-modafinil): VAS=2. Subject reported that it took longer than usual to fall asleep. Subject reported that he usually falls asleep within a few minutes of reclining, but was significantly more alert after taking the contents of this vial (later revealed as containing 200 mg d-modafinil). After falling asleep, subject awoke several times during the night and after awaking, subject reported that it took a long time to fall asleep again.

### Example 4

### Preparation of Film Using Dry Extrusion Techniques

Polyethylene oxide (68 grams, Polyox® WSR N-10) is mixed using mechanical force, and additional ingredients are added during the mixing as follows:

| | |
|---|---|
| d-modafinil | 15 g |
| peppermint | 3.7 g |
| propylene glycol | 3.7 g |
| aspartame | 3.0 g |
| citric acid | 2.6 g |
| Cremphor EL 40 | 3.7 g |
| benzoic acid | 0.05 g |

The temperature is maintained at about 70° C. and blended until uniform. The mixture is then forced through an extrusion die to form a film. The film is then cut into dosage forms ready for packaging.

### Example 5

### Fast Dissolving Microparticulates

Fast dissolving, mucoadhesive microparticulate are prepared basically as previously described (Cilurzo et al., Eur. J. Pharm. Sci., 24(4): 355-361 (2005)) and containing Eudragit® or Carbopol® as a mucoadhesive excipient.

Fast dissolving tablets comprising 100 mg doses of d-modafinil are formulated as follows:

| | | |
|---|---|---|
| d-modafinil | 100 | mg |
| powdered mannitol | 425 | mg |
| citric acid | 11 | mg |
| sweetener | 30 | mg |
| glidant | 2 | mg |
| lubricant | 9.75 | mg |
| hydroscopic agent | 52 | mg |
| flavoring agent | 22.75 | mg |
| color | 1.95 | mg |
| total | 655 | mg tablet weight |

Hydroscopic agents useful in the above recipe may include microcrystalline cellulose (AVICEL PH 200, AVICEL PH 101), Ac-Di-Sol (Croscarmelose Sodium), and PVP-XL (a crosslinked polyvinylpyrrolidone), starches, modified starches, polymers, gum (such as arabic or xanthan), and hydroxyalkyl cellulose (e.g., hydroxymethylcellulose, hydroxypropylcellulose, hydroxypropylmethylcellulose).

Tablets are produced using a direct compression method as follows. All of the ingredients, except the lubricant are weighed and combined. Thereafter, the lubricant is added, and the mixture is blended. Tablets of the blended mixture are then produced using a conventional tablet press. The average in vitro disintegration time is less than 30 seconds in deionized water. The tablets rapidly disintegrate in the mouth.

### Example 6

### Example of a Pharmacokinetic Study

A pharmacokinetic study comparing the circulating plasma half life (T_{1/2}) and Tₘₐₓ of sublingually administered d-modafinil compared to ingested d-modafinil or racemic modafinil is conducted by the following protocol. Healthy normal male volunteers are assessed for the plasma level of d-modafinil on three occasions separated by one week. On one of those occasions, the volunteers swallow one tablet of 50 to 200 mg modafinil (PROVIGIL®, racemic mixture of d- and l-modafinil). On another occasion the volunteers swallow 50 to 200 mg of d-modafinil. On the third occasion, the volunteers take 50 to 200 mg of d-modafinil sublingually in one of the forms described above.

Blood samples are collected by putting a small plastic tube (catheter) into a vein in the arm of the volunteers. Plasma sample are taken just prior to (0), 15, 30, 60, 120, 480, and 640 minutes after the volunteers ingest the d-modafinil and again at 24 hours after ingestion. The assessment of d-modafinil is conducted using high performance liquid chromatography (HPLC) as described by Donovan et al. (Ther. Drug Monit., 25(2): 197-202 (2003)).

All patents, applications, and publications cited in the above text are incorporated herein by reference.

Other variations and embodiments of the invention described herein will now be apparent to those of skill in the art without departing from the disclosure of the invention or the claims below.

1. A method to promote or enhance the state of wakefulness, alertness, or central nervous system (CNS) stimulation in a human individual comprising:
administering to a mucosal membrane other than of the gastrointestinal tract of said human individual a composition comprising a modafinil component comprising as an active ingredient d-modafinil and optionally l-modafinil, wherein said modafinil component is greater than 50% by weight d-modafinil and up to 100% by weight d-modafinil, and wherein the proportion of d-modafinil in said modafinil component is selected so as to achieve a circulating half-life of modafinil desired for said individual, said circulating half-life being less than 11 hours.

2. A method to promote or enhance the state of wakefulness, alertness, or central nervous system (CNS) stimulation in a human individual comprising:
administering to a mucosal membrane other than of the gastrointestinal tract of said human individual a composition comprising a modafinil component comprising as an active ingredient d-modafinil and optionally l-modafinil, wherein said modafinil component is greater than 50% by weight d-modafinil and up to 100% by weight d-modafinil, and wherein the proportion of d-modafinil in said modafinil component is selected so as to achieve a period of enhanced wakefulness, alertness, or CNS stimulation of from about 1 hour to less than 10 hours.

3. The method according to claim 1, wherein said modafinil component is greater than 90% by weight of d-modafinil, and said circulating half-life is less than 4 hours.

4. The method according to claim 2, wherein said modafinil component is greater than 90% by weight of d-modafinil.

5. The method according to claim 3 or claim 4, wherein said modafinil component is essentially 100% by weight d-modafinil.

6. The method according to claim 1 or claim 2, wherein the total amount of modafinil present in said modafinil component is from 10 milligrams to 600 milligrams.

7. The method according to claim 6, wherein the total amount of modafinil present in said modafinil component is from 50 milligrams to 200 milligrams.

8. The method according to claim 1 or claim 2, wherein said mucosal membrane is an oral mucosal membrane.

9. The method according to claim 8, wherein said oral mucosal membrane is a sublingual mucosal membrane or a buccal mucosal membrane.

10. The method according to claim 1 or claim 2, wherein said composition further comprises one or more additional agents selected from the group consisting of a pharmaceutically acceptable carrier, a taste-masking agent, a flavoring agent, a drug different from modafinil that affects the central nervous system, an antimicrobial agent, a plasticizing agent, a buffering agent, a lubricating agent, a preservative, an inert filler agent, a hydrogel, a coloring agent, an enhancer of absorption or transport across mucous membranes, and combinations thereof.

11. The method according to claim 1 or claim 2, wherein said composition is in the form of an orally dissolvable film, a fast dissolving tablet, or a mucoadhesive microparticle.

23. The method according to claim 1 or claim 2, wherein the individual operates machinery or a motor vehicle while in the enhanced state of wakefulness, alertness, or CNS stimulation.

24. The method according to claim 23, wherein said motor vehicle is selected from the group consisting of an automobile, a motorcycle, a truck, a boat, a ship, an aircraft, a train, a streetcar, and an armored vehicle.

25. The method according to claim 23, wherein the individual is operating a motor vehicle after work.

26. The method according to claim 23, wherein the individual is operating a motor vehicle after work on a nightshift.

27. The method according to claim 1 or claim 2, wherein the individual is learning new subject matter while in the enhanced state of wakefulness, alertness, or CNS stimulation.

12. A method of enhancing the effectiveness of a neurorehabilitation program to improve or restore an impaired neurological function of a human individual comprising:
(a) administering to a mucosal membrane other than of the gastrointestinal tract of said human individual a composition comprising a modafinil component comprising as an active ingredient d-modafinil and optionally l-modafinil, wherein said modafinil component is greater than 50% by weight d-modafinil and up to 100% by weight d-modafinil,
(b) engaging said human individual who has been administered said composition in a neurorehabilitation program comprising one or more neurostimuli designed to enhance or restore said impaired neurological function,
(c) optionally, permitting said human individual to rest or sleep for a period of time after engaging in said neurorehabilitation program in step (b), followed by repetition of said steps (a) and (b).

13. The method according to claim 12, wherein the proportion of d-modafinil in said composition is adjusted so as to achieve a circulating half-life of modafinil desired for said individual, said circulating half-life being less than 11 hours; and wherein said optional step (c) is performed after substantially complete clearance of modafinil from the circulation of said individual.

14. The method according to claim 13, wherein said modafinil component is greater than 90% by weight d-modafinil and said circulating half-life is less than 4 hours.

15. The method according to claim 14, wherein said modafinil component is essentially 100% by weight d-modafinil.

16. The method according to claim 12, wherein the total amount of modafinil present in said modafinil component is from 10 milligrams to 600 milligrams.

17. The method according to claim 16, wherein the total amount of modafinil present in said modafinil component is from 50 milligrams to 200 milligrams.

18. The method according to claim 12, wherein said mucosal membrane is an oral mucosal membrane.

19. The method according to claim 18, wherein said oral mucosal membrane is a sublingual mucosal membrane or a buccal mucosal membrane.

20. The method according to claim 12, wherein said composition further comprises one or more additional agents selected from the group consisting of a pharmaceutically acceptable carrier, a taste-masking agent, a flavoring agent, a drug different from modafinil that affects the central nervous system, an antimicrobial agent, a plasticizing agent, a buffering agent, a lubricating agent, a preservative, an inert filler agent, a hydrogel, a coloring agent, an enhancer of absorption or transport across mucous membranes, and combinations thereof.

21. The method according to claim 12, wherein said composition is in the form of an orally dissolvable film, a fast dissolving tablet, or a mucoadhesive microparticle.

22. A solid, fast-dissolving, intraorally administrable composition for promoting or enhancing the state of wakefulness, alertness, or central nervous system (CNS) stimulation in a human individual or for enhancing the effectiveness of a neurorehabilitation program to improve or restore an impaired neurological function of a human individual comprising; a modafinil component comprising as an active ingredient d- and optionally l-modafinil and wherein said modafinil component is greater than 50% by weight d-modafinil and wherein said composition is formulated in a rapidly dissolving delivery form for the administration and rapid absorption by the sublingual or buccal mucosa wherein said delivery form is selected from the group consisting essentially of an orally dissolvable film, a fast dissolving tablet, and a mucoadhesive particle.

23. The composition according to claim 22, wherein said modafinil component is greater than 50% by weight and less than 100% by weight d-modafinil.

24. The composition according to claim 23, wherein said modafinil component is greater than 50% and up to 60% d-modafinil.

25. The composition according to claim 23, wherein said modafinil component is greater than 60% and up to 70% d-modafinil.

26. The composition according to claim 23, wherein said modafinil component is greater than 70% and up to 80% d-modafinil.

27. The composition according to claim 23, wherein said modafinil component is greater than 80% and up to 90% d-modafinil.

28. The composition according to claim 23, wherein said modafinil component is greater than 90% and up to 95% d-modafinil.

29. The composition according to claim 23, wherein said modafinil component is greater than 95% and up to 99% d-modafinil.

30. The composition according to claim 22, wherein said modafinil component is essentially 100% by weight d-modafinil and essentially 0% by weight l-modafinil.

31. The composition according to claim 22, wherein said modafinil component is greater than 0% by weight l-modafinil and less than 50% by weight l-modafinil.

32. The composition according to claim 22, wherein the total amount of modafinil present in said modafinil component is from about 10 milligrams to about 600 milligrams.

33. The composition according to claim 32, wherein the total amount of modafinil present in said modafinil component is from about 50 mg to about 200 mg.

34. The composition according to claim 22, further comprising an agent selected from the group consisting of plasticizing agent, pharmaceutically acceptable carrier, a taste-masking agent, a flavoring agent, a drug different from modafinil that affects the central nervous system, an antimicrobial agent, a buffering agent, a lubricating agent, a preservative, an inert filler agent, a hydrogel, a coloring agent, an enhancer of absorption or transport across mucous membranes, and combinations thereof.
